(19) United States
(12) Patent Application Publication
(10) Pub. No.: US 2016/0091583 A1
(43) Pub. Date: Mar. 31, 2016

(54) Patient-Specific Estimation of Specific Absorption Rate

(71) Applicant: Siemens Aktiengesellschaft, Munchen (DE)

(72) Inventors: Haris Saybasili, Chicago, IL (US); John Kirsch, Charlestown, MA (US); Sven Zuehlsdorff, Westmont, IL (US)

(21) Appl. No.: 14/502,087

(22) Filed: Sep. 30, 2014

(51) Int. Cl. G01R 33/54; G01R 33/28; G01R 33/30; A61B 5/0408; A61B 5/00

(52) U.S. Cl. CPC G01R 33/543; A61B 5/0408; A61B 5/0013; A61B 5/742; G01R 33/30; G01R 33/28; A61B 2560/0487

(57) ABSTRACT

A method for optimizing Specific Absorption Rate (SAR) estimation using a Magnetic Resonance Imaging (MRI) Scanner includes detecting movement of a table holding a patient into a bore of the MRI Scanner and, while the table is moving into the bore, performing an MRI scan of the patient to acquire a multi-slice multi-dimensional MRI dataset of an anatomical region of interest of the patient. The multi-slice multi-dimensional MRI dataset is processed to obtain a three-dimensional model corresponding to the patient's body geometry. Then, a patient-optimized SAR estimation is calculated using the three-dimensional model of the patient's body geometry.

### TECHNICAL FIELD

The present invention relates generally to methods, systems, and apparatuses for using Magnetic Resonance Imaging (MRI) techniques to provide a patient-specific estimation of specific absorption rate (SAR) based features such as, for example, the geometry of the patient and the internal structure of the region of interest being scanned.

### BACKGROUND

Magnetic Resonance Imaging (MRI) is a non-invasive medical imaging technique that utilizes magnetization to visualize soft tissue. The object to be imaged is placed in a very strong static magnetic field. Then, time-varying radio frequency (RF) pulses and magnetic gradient field pulses are applied to enable spatial encoding and provide the ability to distinguish different tissue types after reconstructing images. In comparison to other anatomical imaging techniques, MRI is comparably safe due to its lack of ionizing radiation. However, there are still some risks inherent in MRI applications. For example, the transmission of time-varying RF pulses may induce electrical currents that may result in tissue heating. Unfortunately, it is not practical to measure the heating during imaging with conventional systems. Instead, another approach is used to monitor patient's safety: Specific Absorption Rate (SAR).

SAR measures the rate at which the energy is absorbed by the patient's body during imaging. Specifically, prior to each MRI scan, SAR is estimated based on factors such as the MRI imaging protocol being employed, the body region being imaged, and habitus of the body. Imaging may only be performed if SAR estimation is below limits defined by regulatory bodies such as the Food and Drug Administration (FDA) and it is determined to be safe. Otherwise, acquisition parameters must be adjusted accordingly. In some circumstances, if the predicted SAR is higher than the regulatory limits but lower than a specific threshold, an imaging scan may be subject to a careful risk-benefit analysis of the physician. No scans should be performed if the estimated SAR is larger than the maximum safety levels.

SAR estimates are typically based on simulated human models that are based on a limited set of parameters such as height, weight, age, and gender. However, as noted above, SAR significantly depends on other factors such as the body region being imaged and habitus of the body. Ignoring these factors during estimation of SAR provides an inaccurate, potentially unsafe estimation that may result in compromises in the scan protocol and possibly sub-optimal image quality. Underestimation of the SAR may cause a significant health risk for the patient. Conversely, overestimation of SAR may limit the energy to be deposited to suboptimal levels during the scan and, hence, may decrease overall image quality or diagnostic utility. Moreover, as SAR is roughly proportional to the square of the field strength, the accuracy of the SAR predictions becomes even more important for ultra-high field MRI Scanners (e.g., 7T), an emerging technology with significant clinical potential.

Additionally, multi-transmit systems such as parallel transmit arrays have been recently developed to improve homogeneity of the overall transmission field on ultra-high field systems. This is achieved by transmitting multiple, locally controlled, radiofrequency (RF) pulses simultaneously. However, this approach makes it difficult to estimate the SAR levels correctly since the multiple independent excitations from different transmit channels will be superimposed inside the body.

### SUMMARY

Embodiments of the present invention address and overcome one or more of the above shortcomings and drawbacks, by providing methods, systems, and apparatuses that improve Specific Absorption Rate (SAR) estimates by measuring patent-specific features such as geometry directly on the scanner prior to the imaging session. As a result, both patient safety and image quality could be improved on standard and high field Magnetic Resonance Imaging (MRI) Scanners.

According to some embodiments of the present invention, a method for optimizing SAR estimation using a MRI Scanner includes detecting movement of a table holding a patient into a bore of the MRI Scanner and, while the table is moving into the bore, performing an MRI scan to acquire a multi-slice multi-dimensional MRI dataset of an anatomical region of interest of the patient. The MRI dataset is processed to obtain a three-dimensional model corresponding to the patient's body geometry. Then, a patient-optimized SAR estimation is calculated using the model. An MRI study may then be performed using the patient-optimized SAR estimation. In some embodiments, one or more tissue properties of the anatomical region of interest are identified based on the three-dimensional model. These tissue properties may then be used in the calculation of the patient-optimized local and whole body SAR estimation.

Various enhancements, modification, additions, and/or refinements, may be made to the aforementioned method according to some embodiments of the present invention. For example, in one embodiment, the MRI scan is performed using a noise reduction process designed to minimize acoustic noise generated by the MRI Scanner during the MRI scan. The noise reduction process may, for example, optimize gradient switching of the MRI Scanner during the MRI scan. In another embodiment, an initial SAR estimation is determined using a default human body model prior to performing the MRI scan. Then, the default human body model is updated using the three-dimensional model of the patient's body geometry. In another embodiment, the acquisition of the multi-slice multi-dimensional MRI dataset utilizes one or more measurement devices placed on the patient such as, for example, acquisition coils and/or electrocardiogram electrodes.

In some embodiments, of the present invention, the MRI scan utilizes an ultra low-SAR pulse sequence designed to produce SAR levels below a peak recommended value in the anatomical region of interest. The peak recommended values may vary. For example, in one embodiment the peak recommended value is 1.5 Watts per Kilogram, while in another embodiment, the peak recommended value is 0.5 Watts per Kilogram.

The aforementioned method can be provided as part of a device, apparatus or article of manufacture. For example, in one embodiment, an article of manufacture for optimizing Specific Absorption Rate (SAR) estimation using a Magnetic Resonance Imaging (MRI) Scanner includes a non-transitory, tangible computer-readable medium holding computer-executable instructions for performing the aforementioned method.

According to other embodiments of the present invention, a system is used for optimizing SAR estimation. This system includes an MRI Scanner with a table configured to hold a patient and a bore configured to receive the table. The system also includes an image processing computer configured to detect movement of the table into the bore and to use the MRI Scanner to perform an MRI scan of the patient while the table is moving to acquire a multi-slice multi-dimensional MRI dataset of an anatomical region of interest of the patient. The image processing computer is further configured to process the MRI dataset to obtain a three-dimensional model corresponding to the patient's body geometry and to calculate a patient-optimized SAR estimation using the model.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 provides an overview of a system that may be used in performing patient-optimized SAR estimation, according to some embodiments of the present invention;

FIG. 2 shows a process for determining a patient-optimized SAR estimation, according to some embodiments of the present invention;

FIG. 3 provides images of three-dimensional body geometry generated from multi-slice multi-dimensional images, according to some embodiments of the present invention; and

FIG. 4 illustrates an exemplary computing environment within which embodiments of the invention may be implemented

### DETAILED DESCRIPTION

The present invention relates generally to methods, systems, and apparatuses for optimizing Specific Absorption Rate (SAR) estimations on a per-patient basis based on an MRI scan of negligible SAR of the patient's body. Current SAR estimation models often overestimate SAR and, as a result, total energy is kept lower than ideal levels during MRI data acquisition. As a result, the overall image quality and diagnostic utility may be decreased. On the other hand, inaccuracies in estimation models may also underestimate SAR increasing risk to the patient. Using the techniques described herein, a patient's anatomical features such as geometry and tissue composition are determined as part of the SAR estimation process, thereby improving both patient safety and overall image quality.

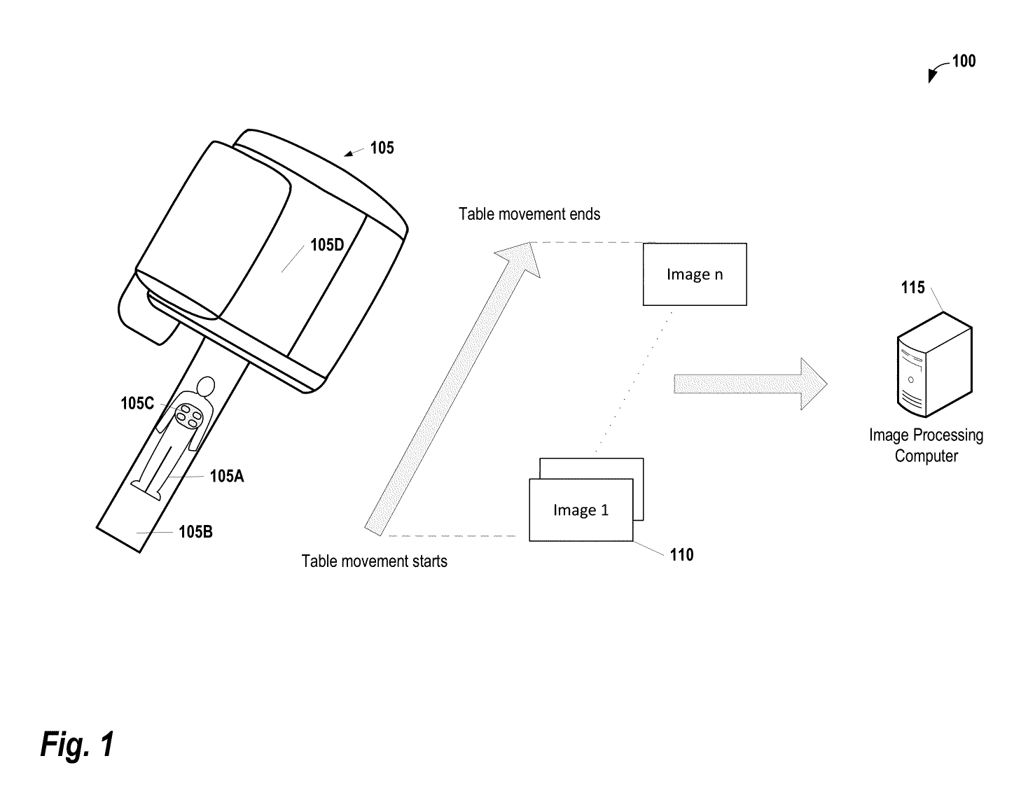

FIG. 1 provides an overview of a system 100 that may be used in performing patient-optimized SAR estimation, according to some embodiments of the present invention. Briefly, the system 100 is used to generate a three-dimensional model of a patient's body for use in SAR estimation prior to an MRI study. During the preparation phase of the study, the Patient 105A is positioned on the Scanner Table 105B of the MRI Scanner 105. Measurement Devices 105C (e.g., electrocardiogram electrodes, acquisition coils, etc.) are positioned on the Patient 105A over the region of interest (i.e., the region being scanned). Following that, the region of interest on the body landmarked and the Scanner Table 105B is sent into the Bore 105D of the MRI Scanner 105. The term landmarking, as used herein, refers to aligning the patient with the isocenter of the MRI Scanner 105. In some embodiments, landmarking is performed using an alignment light projected from the entrance of the Bore 105D. In other embodiments, an external laser system (not shown in FIG. 1) may be used.

The Scanner Table 105B moves slowly into the Bore 105D, and stops once the landmarked region is at the isocenter of the Bore 105D. In some embodiments, the Scanner Table 105B does not stop at the isocenter, but rather continues moving to cover a wider region of the body of the Patient 105A. Once full coverage is achieved, the Scanner Table 105B can be returned back to the isocenter. An MRI scan is performed as the Scanner Table 105B is sent into the Bore 105D for use in SAR estimation. In some embodiments, this scan comprises a low (or ultra-low) SAR, fast 2D multi-slice MRI scan. As the scan is performed the MRI Scanner 105 transmits multi-dimensional (e.g., two-dimensional or three-dimensional) images 110 to Image Processing Computer 115 for reconstruction into a three-dimensional model. The data from this three-dimensional model is used to calculate an optimal, patient-specific SAR estimation that can be used during the actual study. In some embodiments, prior to creating the model and calculating the SAR estimation, the Image Processing Computer 115 detects movement of the table and communicates with the MRI Scanner 105 to perform the scan accordingly. It should be noted that imaging modalities other than MRI may also be used to generate the three-dimensional model. For example, in one embodiment, high-precision thermo-nuclear cameras are used.

This example illustrated in FIG. 1 may use a conventional SAR model for estimation with protocol parameters that are known to be well below limits of SAR for all patient scenarios. In some embodiments, the scan is designed to be short enough to finish by the time the technician returns to the MRI control room. One or more “quiet” scan techniques may be used to reduce the amount of MRI acoustic noise produced by the scan. For example, it is known in the art that the rapid switching of gradients during a scan generates loud mechanical vibrations. Thus, in some embodiments, the noise generated by the scan is reduced in software by optimizing gradient switching to provide the best possible gradient trajectory through an intelligent summation of gradients and reduction of the slew rate.

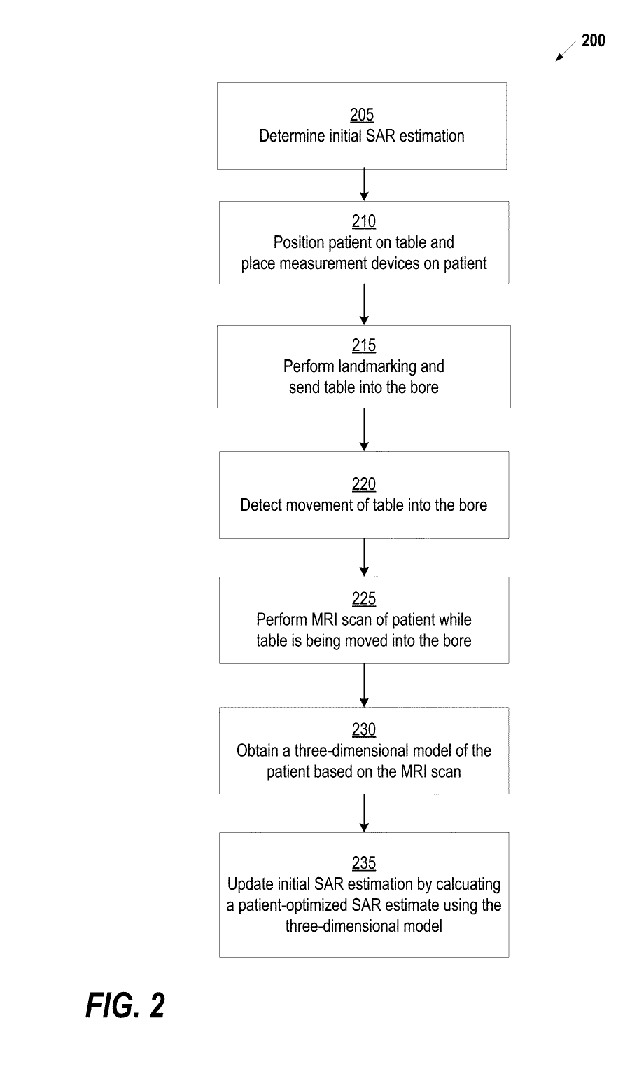

FIG. 2 shows a process 200 for determining a patient-optimized SAR estimation, according to some embodiments of the present invention. This process may be performed, for example, using the system 100 illustrated in FIG. 1. At 205, an initial SAR estimation is determined using a default human body model estimated by relevant patient registration parameters (e.g., age, gender, height, weight). Next, at 210, the patient is positioned on the table of the MRI Scanner and measurement devices are placed on the patient. These measurement devices may include, for example, acquisition coils, electrocardiogram electrodes, and/or other similar devices. Then, at 215, the technician of the MRI Scanner landmarks the region of interest before sending the table into bore of the MRI Scanner.

Continuing with reference to FIG. 2, at 220, the imaging computer detects the movement of the table into the bore of the MRI Scanner. Then, at 225, while the table is moving into the bore, an MRI scan of the patient is performed to acquire MRI data to provide three-dimensional coverage of the patient's body. In some embodiments, this coverage is provided by a multi-slice multi-dimensional MRI dataset of an anatomical region of interest of the patient. In some embodiments, the MRI scan is performing using a noise reduction process designed to minimize acoustic noise generated by the MRI Scanner during the MRI scan (i.e., a “quiet” scan). For example, in one embodiment, this noise reduction process optimizes gradient switching of the MRI Scanner during the MRI scan. In some embodiments, the MRI scan utilizes an ultra low-SAR pulse sequence designed to produce SAR levels well below a peak recommended value in the anatomical region of interest. For example, in some embodiments, the peak recommended value is 1.5 Watts per Kilogram of the patient's body weight (i.e., a “low” SAR scan). In other embodiments, the peak recommended value is 0.5 Watts per Kilogram of the patient's body weight (i.e., an “ultra-low” SAR scan).

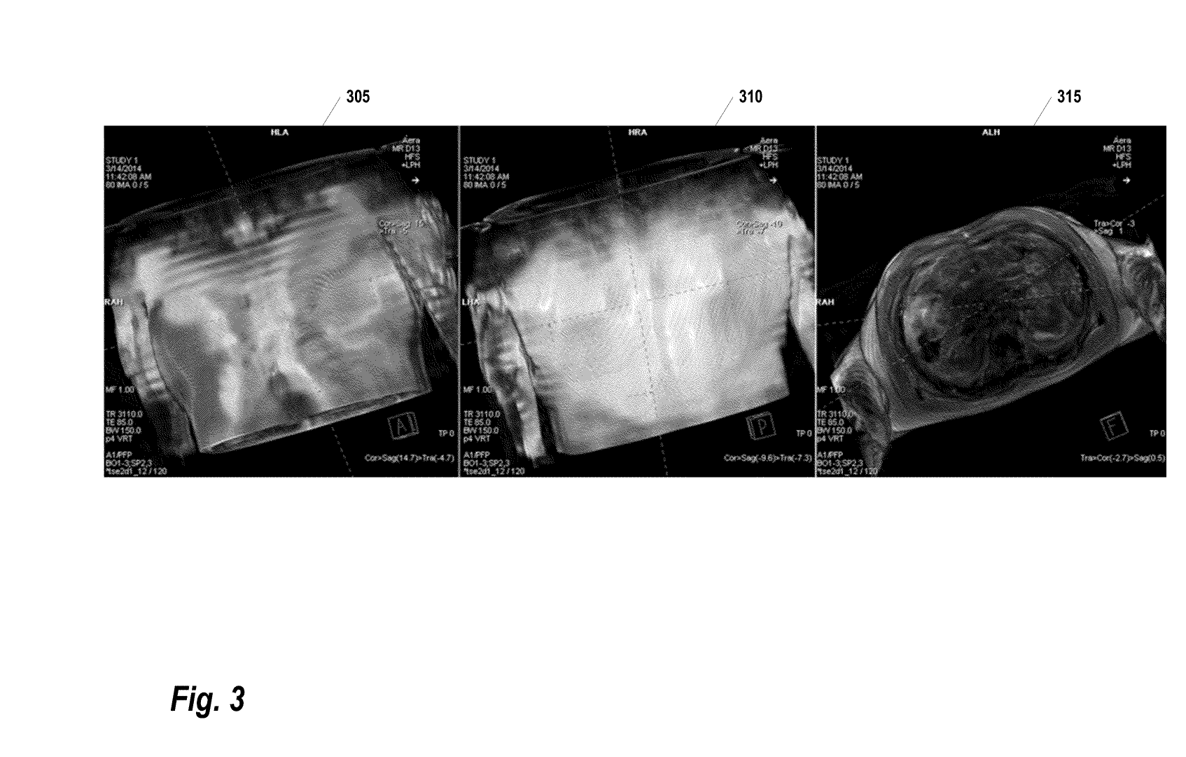

At 230, the multi-slice multi-dimensional MRI dataset acquired at 225 is processed to obtain a three-dimensional model corresponding to the patient's body geometry. Various techniques of determining the three-dimensional model may be used in different embodiments of the present invention. For example, in some embodiments, two-dimensional or three-dimensional images may be acquired and stacked to produce the three-dimensional model. FIG. 3 provides a set of images showing a three-dimensional model developed using such a technique, according to some embodiments of the present invention. The example of FIG. 3 includes images showing the model in a front orientation 305, a back orientation 310, and a bottom orientation 315. Returning to FIG. 2, at 235, the three-dimensional model is used to update the default body model utilized in the initial SAR estimation at 205 and to provide a patient-optimized SAR estimation. Once the process 200 is complete, an MRI study may then be performed using this estimation.

Various techniques may be used for calculating the SAR estimate based on the three-dimensional model. For example, in some embodiments, conventional estimation algorithms may be used with the three-dimensional model pre-processed to meet the input requirements of the respective algorithms. In other embodiments, enhanced SAR estimation algorithms may be employed which take advantage of the additional information that may be available in the model. For example, different tissue components have different electrical properties which, in turn, may result in different heat distributions. Thus, knowledge of the tissue type gleaned from the three-dimensional model may be included as an input to the estimation algorithm to provide more accurate representation of the true local SAR estimations of the region of interest.

Moreover, conventional systems for performing MRI scans have no knowledge of where the patient's body is in relation to the walls of the bore. Although the RF transmission field from the body coil used in the scan is designed to be homogenous across the whole inner volume of the bore, in reality the RF exposure can be extremely high around the edges of the bore. Any portion of the patient's body which touches the bore could be significantly warmed, or in worst case, burned. As a result, technicians administering the scan typically try to position the patient as far away from the sides of the bore as possible. However, the technician has no way of knowing in real-time (or near real-time) whether that patient's body is actually touching the side of the bore. Using the techniques described herein, the geometry of the patient can be directly ascertained via the three-dimensional model. The patient's geometry may then be compared to the geometry of the bore to provide a more accurate assessment of the patient position within the scanner.

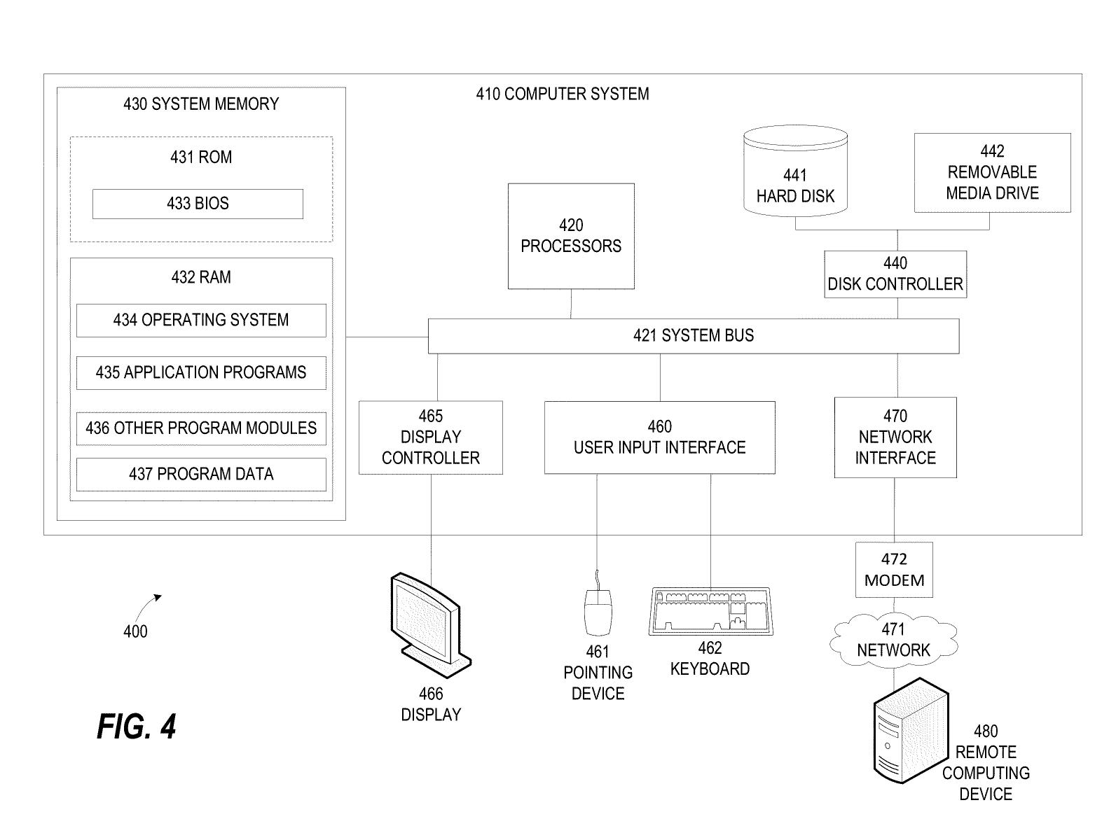

FIG. 4 illustrates an exemplary computing environment 400 within which embodiments of the invention may be implemented. For example, computing environment 400 may be used to implement one or more components of system 100 shown in FIG. 1 such as Image Processing Computer 115. Computers and computing environments, such as computer system 410 and computing environment 400, are known to those of skill in the art and thus are described briefly here.

As shown in FIG. 4, the computer system 410 may include a communication mechanism such as a system bus 421 or other communication mechanism for communicating information within the computer system 410. The computer system 410 further includes one or more processors 420 coupled with the system bus 421 for processing the information.

The processors 420 may include one or more central processing units (CPUs), graphical processing units (GPUs), or any other processor known in the art. More generally, a processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and be conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

Continuing with reference to FIG. 4, the computer system 410 also includes a system memory 430 coupled to the system bus 421 for storing information and instructions to be executed by processors 420. The system memory 430 may include computer readable storage media in the form of volatile and/or nonvolatile memory, such as read only memory (ROM) 431 and/or random access memory (RAM) 432. The system memory RAM 432 may include other dynamic storage device(s) (e.g., dynamic RAM, static RAM, and synchronous DRAM). The system memory ROM 431 may include other static storage device(s) (e.g., programmable ROM, erasable PROM, and electrically erasable PROM). In addition, the system memory 430 may be used for storing temporary variables or other intermediate information during the execution of instructions by the processors 420. A basic input/output system 433 (BIOS) containing the basic routines that help to transfer information between elements within computer system 410, such as during start-up, may be stored in system memory ROM 431. System memory RAM 432 may contain data and/or program modules that are immediately accessible to and/or presently being operated on by the processors 420. System memory 430 may additionally include, for example, operating system 434, application programs 435, other program modules 436 and program data 437.

The computer system 410 also includes a disk controller 440 coupled to the system bus 421 to control one or more storage devices for storing information and instructions, such as a magnetic hard disk 441 and a removable media drive 442 (e.g., floppy disk drive, compact disc drive, tape drive, and/or solid state drive). The storage devices may be added to the computer system 410 using an appropriate device interface (e.g., a small computer system interface (SCSI), integrated device electronics (IDE), Universal Serial Bus (USB), or FireWire).

The computer system 410 may also include a display controller 465 coupled to the system bus 421 to control a display 466, such as a cathode ray tube (CRT) or liquid crystal display (LCD), for displaying information to a computer user. The computer system includes an input interface 460 and one or more input devices, such as a keyboard 462 and a pointing device 461, for interacting with a computer user and providing information to the one or more processors 420. The pointing device 461, for example, may be a mouse, a light pen, a trackball, or a pointing stick for communicating direction information and command selections to the one or more processors 420 and for controlling cursor movement on the display 466. The display 466 may provide a touch screen interface which allows input to supplement or replace the communication of direction information and command selections by the pointing device 461.

The computer system 410 may perform a portion or all of the processing steps of embodiments of the invention in response to the one or more processors 420 executing one or more sequences of one or more instructions contained in a memory, such as the system memory 430. Such instructions may be read into the system memory 430 from another computer readable medium, such as a magnetic hard disk 441 or a removable media drive 442. The magnetic hard disk 441 may contain one or more datastores and data files used by embodiments of the present invention. Datastore contents and data files may be encrypted to improve security. The processors 420 may also be employed in a multi-processing arrangement to execute the one or more sequences of instructions contained in system memory 430. In alternative embodiments, hard-wired circuitry may be used in place of or in combination with software instructions. Thus, embodiments are not limited to any specific combination of hardware circuitry and software.

As stated above, the computer system 410 may include at least one computer readable medium or memory for holding instructions programmed according to embodiments of the invention and for containing data structures, tables, records, or other data described herein. The term “computer readable medium” as used herein refers to any medium that participates in providing instructions to the one or more processors 420 for execution. A computer readable medium may take many forms including, but not limited to, non-transitory, non-volatile media, volatile media, and transmission media. Non-limiting examples of non-volatile media include optical disks, solid state drives, magnetic disks, and magneto-optical disks, such as magnetic hard disk 441 or removable media drive 442. Non-limiting examples of volatile media include dynamic memory, such as system memory 430. Non-limiting examples of transmission media include coaxial cables, copper wire, and fiber optics, including the wires that make up the system bus 421. Transmission media may also take the form of acoustic or light waves, such as those generated during radio wave and infrared data communications.

The computing environment 400 may further include the computer system 410 operating in a networked environment using logical connections to one or more remote computers, such as remote computer 480. Remote computer 480 may be a personal computer (laptop or desktop), a mobile device, a server, a router, a network PC, a peer device or other common network node, and typically includes many or all of the elements described above relative to computer system 410. When used in a networking environment, computer system 410 may include modem 472 for establishing communications over a network 471, such as the Internet. Modem 472 may be connected to system bus 421 via user network interface 470, or via another appropriate mechanism.

Network 471 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 410 and other computers (e.g., remote computing 480). The network 471 may be wired, wireless or a combination thereof. Wired connections may be implemented using Ethernet, Universal Serial Bus (USB), RJ-6, or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 471.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically, wholly or partially in response to user command. An activity (including a step) performed automatically is performed in response to one or more executable instructions or device operation without user direct initiation of the activity.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112, sixth paragraph, unless the element is expressly recited using the phrase “means for.”

We claim:

1. A method for optimizing Specific Absorption Rate (SAR) estimation using a Magnetic Resonance Imaging (MRI) Scanner, the method comprising:
detecting movement of a table holding a patient into a bore of the MRI Scanner;
while the table is moving into the bore, performing an MRI scan of the patient to acquire a multi-slice multi-dimensional MRI dataset of an anatomical region of interest of the patient;
processing the multi-slice multi-dimensional MRI dataset to obtain a three-dimensional model corresponding to body geometry of the patient; and
calculating a patient-optimized SAR estimation using the three-dimensional model of the body geometry of the patient.

2. The method of claim 1, further comprising:
performing an MRI study using the patient-optimized SAR estimation.

3. The method of claim 1, wherein the MRI scan is performed using a noise reduction process designed to minimize acoustic noise generated by the MRI Scanner during the MRI scan.

4. The method of claim 3, wherein the noise reduction process optimizes gradient switching of the MRI Scanner during the MRI scan.

5. The method of claim 1, further comprising:
calculating an initial SAR estimation using a default human body model prior to performing the MRI scan; and
updating the default human body model using the three-dimensional model of the body geometry of the patient.

6. The method of claim 1, wherein acquisition of the multi-slice multi-dimensional MRI dataset utilizes one or more measurement devices placed on the patient.

7. The method of claim 6, wherein the one or more measurement devices comprise one or more of acquisition coils and electrocardiogram electrodes.

8. The method of claim 1, wherein the MRI scan utilizes an ultra low-SAR pulse sequence designed to produce SAR levels below a peak recommended value in the anatomical region of interest.

9. The method of claim 8, wherein the peak recommended value is 1.5 Watts per Kilogram.

10. The method of claim 8, wherein the peak recommended value is 0.5 Watts per Kilogram.

11. The method of claim 1, further comprising:
identifying one or more tissue properties of the anatomical region of interest based on the three-dimensional model of the body geometry of the patient,
wherein calculation of the patient-optimized SAR estimation is based on the one or more tissue properties, and
wherein the patient-optimized SAR estimation comprises a local and whole body SAR estimation.

12. An article of manufacture for optimizing Specific Absorption Rate (SAR) estimation using a Magnetic Resonance Imaging (MRI) Scanner, the article of manufacture comprising a non-transitory, tangible computer-readable medium holding computer-executable instructions for performing a method comprising:
detecting movement of a table holding a patient into a bore of the MRI Scanner;
while the table is moving into the bore, performing an MRI scan of the patient to acquire a multi-slice multi-dimensional MRI dataset of an anatomical region of interest of the patient;
processing the multi-slice multi-dimensional MRI dataset to obtain a three-dimensional model corresponding to body geometry of the patient; and
calculating a patient-optimized SAR estimation using the three-dimensional model of the body geometry of the patient.

13. The article of manufacture of claim 12, wherein the MRI scan is performed using a noise reduction process designed to minimize acoustic noise generated by the MRI Scanner during the MRI scan.

14. The article of manufacture of claim 13, wherein the noise reduction process optimizes gradient switching of the MRI Scanner during the MRI scan.

15. The article of manufacture of claim 12, wherein the method further comprises:
calculating an initial SAR estimation using a default human body model prior to performing the MRI scan; and
updating the default human body model using the three-dimensional model of the body geometry of the patient.

16. The article of manufacture of claim 12, wherein the MRI scan utilizes a low-SAR pulse sequence designed to produce SAR levels below a peak recommended value in the anatomical region of interest.

17. The article of manufacture of claim 12, wherein the method further comprises:
identifying one or more tissue properties of the anatomical region of interest based on the three-dimensional model of the body geometry of the patient,
wherein calculation of the patient-optimized SAR estimation is based on the one or more tissue properties.

18. A system for optimizing Specific Absorption Rate (SAR) estimation, the system comprising:
an MRI Scanner comprising:
a table configured to hold a patient, and
a bore configured to receive the table; and
an image processing computer configured to:
detect movement of the table into the bore,
use the MRI Scanner to perform an MRI scan of the patient while the table is moving into the bore thereby acquiring a multi-slice multi-dimensional MRI dataset of an anatomical region of interest of the patient,
process the multi-slice multi-dimensional MRI dataset to obtain a three-dimensional model corresponding to body geometry of the patient, and
calculate a patient-optimized SAR estimation using the three-dimensional model of the body geometry of the patient.

19. The system of claim 18, wherein the image processing computer uses a noise reduction process designed to minimize acoustic noise generated by the MRI Scanner during the MRI scan.

20. The system of claim 18, wherein the image processing computer is configured to use the MRI Scanner to perform the MRI scan with an ultra low-SAR pulse sequence.
